(19) United States
(12) Patent Application Publication
(10) Pub. No.: US 2013/0053433 A1
(43) Pub. Date: Feb. 28, 2013

(54) FATTY ACID DERIVATIVES AND ANALOGS OF DRUGS

(71) Applicants: Moo J. Cho, Chapel Hill, NC (US); Michael Hackett, Chapel Hill, NC (US)

(72) Inventors: Moo J. Cho, Chapel Hill, NC (US); Michael Hackett, Chapel Hill, NC (US)

(73) Assignee: The University of North Carolina at Chapel Hill, Chapel Hill, NC (US)

(21) Appl. No.: 13/520,289

(22) PCT Filed: Jan. 5, 2011
(86) PCT No.: PCT/US11/20221

§ 371 Date: Nov. 8, 2012

Related U.S. Application Data
(60) Provisional application No. 61/292,696, filed on Jan. 6, 2010.

(51) Int. Cl. A61K 31/337; A61P 35/00; C07D 305/14

(52) U.S. Cl. 514/449; 549/510

(57) ABSTRACT

The present disclosure is directed to fatty acid-drug conjugates, the preparation and use thereof and long-chain (C₁₀₋₂₅), both saturated and unsaturated, fatty acids and anhydrides useful for preparing drug conjugates.

### FEDERALLY SPONSORED RESEARCH OR DEVELOPMENT

This invention was made with government support under National Institutes of Health, federal grant CA126825. The government has certain rights in the invention.

### FIELD OF THE INVENTION

The present disclosure is directed to fatty acid derivatives and analogs of drugs, in particular those that contain malonic, succinic, and glutaric acids with a single C₁₀₋₂₅ alkyl chain.

### BACKGROUND OF THE INVENTION

Albumin of MW 66 kDa is the most abundant protein in the body, providing osmotic pressure to blood vessel against hydrostatic pressure from the heart. It also serves as a natural carrier for a variety of xenobiotics as well as water-insoluble endogenous substances such as fatty acids. For instance, paclitaxel (PTX) binds albumin with dissociation constant K_{d} of ˜10⁻⁵ M (Paal, Muller et al. 2001) while stearic acid with K_{d} in the range of μM to nM depending on total fatty acid concentration (Curry, Brick et al. 1999). Its serum concentration is close to 40 mg/ml (4% or 0.61 mM) while it is ˜16 mg/ml (1.6% or 0.24 mM) in the interstitial tissues. Since albumin provides six rather specific binding sites for fatty acid, the effective concentration in terms of fatty acid-binding site is much higher. In short, the protein acts as a sponge for hydrophobic xenobiotics and 99.9% of all fatty acids in the serum are in the state of bound to albumin.

While structurally and functionally quite different from immunoglobulin IgG of MW 150 kDa, there are many similarities between the two major proteins in kinetics of synthesis and catabolism. IgG is second only to albumin in systemic abundance at ˜12 mg/ml. The size of these proteins makes them candidates for the so-called enhanced permeability and retention (EPR) effect proposed by Maeda (Maeda 2001; Tanaka, Shiramoto et al. 2004). Briefly, these molecules move mainly by solvent drag (i.e., convection) through the bloodstream. When approaching a tumor, the pressure gradient pushes these macromolecules through a leaky vasculature into the tumor periphery. The tumor core is of higher fluid pressure preventing any convective penetration deeper into the tumor (Jain 2005). With no ability to move back to the circulation or deeper into the tumor, these macromolecules would ordinarily be drained to the lymphatic system, which is malfunctioning in inner necrotic tumor tissue. It was thus proposed by Stehle that these macromolecules are macropinocytosed into the tumor periphery where they are degraded for nutrition for tumor growth (Stehle, Wunder et al. 1999). As the concentration of albumin drops in the vasculature, the liver synthesizes more to maintain the steady-state 0.6 mM systemic concentration. It is thus not surprising to observe that the tumor is where the majority of albumin degradation occurs (Stehle, Wunder et al. 1998). Increased vascular permeability is also commonly observed in various inflammatory diseases. As such, EPR-mediated accumulation of albumin and IgG in pathologic sites is expected as seen with synthetic nanoparticulates (Sandanaraj, Gremlich et al. 2009).

How humans maintain such high concentrations of albumin and IgG has remained a mystery until the recent discovery that FcRn or Brambell Receptor protects these two major proteins (Anderson, Chaudhury et al. 2006; Kim, Bronson et al. 2006). This receptor recognizes both proteins under acidic conditions of late endosome, where it binds them triggering endosomal recycling back to the surface. The neutral pH of the serum causes dissociation and the intact proteins return to the circulation. This protection manifests in an astounding characteristic for these macromolecules: the half-lives of albumin and IgG are 19 and 21 days, respectively. In summary, albumin is produced in the liver as the most abundant protein in the body. It binds fatty acids with very high affinity and capacity, circulates for an unparalleled length of time and passively targets developing tumors.

There are at least two ways to exploit albumin as a drug carrier. One is direct chemical conjugation of drug molecules to the protein hoping that free drug will be somehow released (Kratz, Muller-Driver et al. 2000). In this case, use of albumin is no different from any synthetic macromolecules since the protein loaded with a large number of drug molecules would no longer behave as native albumin. The second approach is derivatizing a drug with a molecule that increases the drug's affinity for albumin. An example is an ibuprofen conjugate of oligonucleotides in which the PK of the latter changes dramatically due to ibuprofen binding to albumin (Manoharan, Inamati et al. 2002).

Paclitaxel (PTX) is currently the drug of choice in treating breast, ovarian and lung cancers. PTX is extremely insoluble in water and thus many formulations have been attempted to solubilize the drug and to improve its pharmacokinetics (PK) with marginal success (Singh and Dash, 2009). Taxol® (Bristol-Myers Squibb, Princeton, N.J.), in which PTX was solubilized with the surfactant polyoxyethylated castor oil (Cremophor EL) and absolute ethanol, still suffers from poor solubility (<1.2 mg/mL after reconstitution), requires a slow 3-hr infusion and often leads to hypersensitivity due to the large amounts of Cremophor EL present. The side effects include neutropenia and peripheral neuropathy. These reactions had to be preempted with the administration of steroids prior to treatment.

In the second-generation formulation Abraxane® (Abraxis Bioscience, LLC, Los Angeles, Calif.), PTX exists as a complex with albumin. Processing involves denaturing albumin under high pressure in an organic solvent that dissolves PTX. The solvent is then removed to result in the product. When the solid powder is reconstituted in water, submicron particles of about 130 nm are produced at a concentration 2 to 10 mg/ml. The manufacturer suggests that the nanoparticle is transcytosed by gp60 into the tumor periphery although this hypothesis has not been experimentally established (non-peer reviewed journal, Drug Delivery Report Winter 07/08). Due to the albumin denaturation in the formulation, its recognition by gp60 and/or FcRn is questionable and certainly will not enjoy albumin's long half-life.

Insulin-containing product Detemir® is an acylated insulin. Insulin is a natural peptide hormone for the regulation of serum concentrations of glucose. Diabetics can become resistant to insulin or lack sufficient levels of insulin to regulate their glucose levels. These diabetics will take insulin shots, typically three times a day. The purpose of a long circulating insulin molecule would be to maintain basal concentrations of the peptide over the course of the day thus requiring only a single shot. This insulin molecule has a fatty acid attached at the terminus of one of the chains which will allow the peptide to be anchored on albumin. (Kurtzhals, Havelund et al., 1995).

Nucleic acid-lipid conjugates are known. This technology involved the modification of a new class of oligonucleotide drugs with fatty acids or sterols. These modified nucleic acids were shown to retain activity and were also shown to bind albumin as well as lipoprotein particles, depending on the modification. Again, the chemistry involved in the synthesis is tailored to the drug, usually acylation, abolishing the negative charge on the fatty acid. The charge on the carboxylated sterols is neutralized as well, however, only the fatty acid derivatives are shown to appreciably bind albumin (Wolfrum, Shi, et al., 2007).

A gemcitabine conjugated with squalenic acid is known (Couvreur, Stella, et al. 2006). This allows the gemcitabine molecules to aggregate to form micelles or micelle-like particles. There is no mention of albumin binding, but the ramified nature of the fatty acid would more than likely make this conjugate better suited for lipoprotein binding as opposed to albumin. It would likely result in a conjugate having significantly decreased circulation time. A stearic acid (18 carbon fatty acid) derivative of gemcitabine is known. The conjugate was processed to liposomes. The latter is submicron lipid vesicles that are spontaneously formed when phospholipids are suspended in aqueous media. The incorporation of the conjugate into liposomes would likely yield poor half-lives compared to binding albumin.

Derivatizing the hydrophobic drug PTX with the ω-3 docosahexaenoic acid yields the drug Taxoprexin®. (Wolff, Donehower, et al. 2003). This lipid is a natural nutrient believed to be taken up more commonly in tumors. The formulation of this drug is Taxol® like in that it is formulated in Cremophor EL and ethanol then diluted into saline. This formulation increases the solubility of the prodrug in the formulation; however, it does not eliminate the side effects of the Cremophor EL including neutropenia and hypersensitivity. It will still exhibit similar toxicities to Taxol® and have a poor half-life, just like Taxol®. Due to the structure of the lipid, it is more likely to be an low and high density liporoteins (LDL/HDL) binder than albumin thus it will still not enjoy the same increased half-life even if formulated for a natural carrier.

Doxorubicin conjugated with alpha-linolenic acid is similar to Taxoprexin® in that it utilizes the tumor for digestion of natural ω-3 fatty acids (Huan, Zhou, et al. 2009). The hydrophilic nature, specifically the cationic ammonium charge on the sugar, hinders uptake of the drug. This is overcome by the conjugation as it neutralizes the charge and makes the drug much more prone to partition into membranes of cells allowing it to move into cells by simple diffusion. There are many other formulations containing doxorubicin. They are not targeted, neither passively nor actively, to the tumor and thus the severe cardiotoxicity of the drug is expected to endure. Furthermore, with no carrier, the PK of the drug would not be expected to be changed appreciably precluding a decrease in toxicity or increase in efficacy.

Certain 3-substituted glutaric acids and their anhydrides are known (Poldy Peakall, and Barrow, 2008). However, only relatively short chain derivatives are disclosed in the synthesis of pheromones. In this patent application, the short-chain glutaric anhydrides are not used in any manner other than as a stepping stone in the synthesis of pheromones.

In summary, the need exists for a molecule that is capable of modifying a drug's affinity for albumin thereby creating a drug conjugate with desirable biopharmaceutical properties including serum PK with a long circulatory half-life and targeted delivery to a pathological site.

### SUMMARY OF THE INVENTION

Compositions and methods for covalently coupling a polycarboxylic fatty acid and a drug molecule to form a fatty acid-drug conjugate are described. The conjugates are useful for increasing solubility of the drug as well as targeting the drug during therapy to solid tumors in cancer treatment or inflamed joint in arthritis. In one embodiment, the present disclosure is directed to long-chain fatty acid with two or more closely located —COOH groups. The —COOH group in the drug conjugate can be other inorganic acids such as nitrate, sulfate, sulfonate, phosphate, and their derivatives. One —COOH group in these reagents can be covalently coupled to a drug. When conjugated in this way to a drug, the fatty acid molecule contains a free carboxylic acid or other inorganic acid anion(s). These long-chain fatty acid-drug conjugates can have improved biopharmaceutical properties such that a high therapeutic index would be realized.

Another embodiment is directed to long-chain fatty acid molecule-drug conjugates as described herein.

Another embodiment is directed to the use of long-chain fatty acid molecules to prepare fatty acid-drug conjugates.

Another embodiment is directed to the use of the long-chain fatty acid-drug conjugates to increase solubility of the drug.

### BRIEF DESCRIPTION OF THE DRAWINGS

FIG. 1 depicts the solubility of the conjugate increases as the concentration of serum albumin increases. This dependence suggests an interaction between the two. This experiment was done in PBS using crystalline conjugate and we found it is dissolution limited thus it will take forever to equilibrate. To remedy this, we dissolved the PDG-PTX conjugate in t-BuOH and added it slowly to a stirred solution of serum albumin which allowed us to saturate the albumin and achieve significantly higher concentrations (˜825 μM PDG-PTX in a 1% HSA solution)

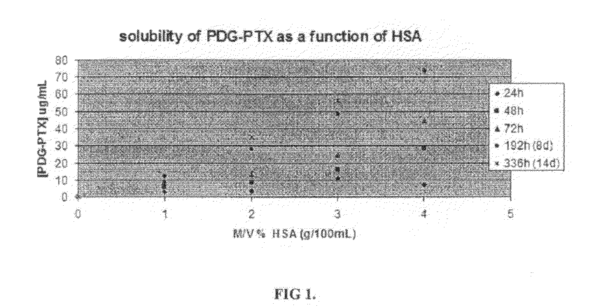

FIG. 2 depicts a theoretical model of how the conjugate will orient itself when bound to serum albumin. Note that the conjugate still carries a carboxylate anion that can enhance binding to the protein via electrostatic interaction with cationic amino acid residues in the periphery of binding pocket. The dicarboxylic acid in this example is stearic acid (C₁₈H₃₆O₂) that contains acetic acid at the α-carbon. One of the two carboxylic acids is coupled to PTX via an ester bond (brown). This ester bond is expected to be cleaved releasing free PTX either in the vicinity of tumor tissue or inside the tumor cell after internalization. Although it is a possibility that the derivative itself is pharmacologically as active as PTX just like docetaxel (Crown, O'Leary et al. 2004), the derivative may also behave as a prodrug.

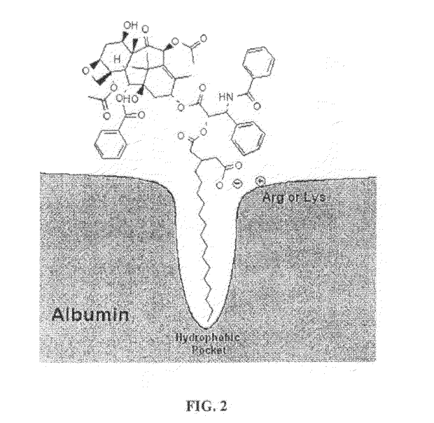

FIG. 3 depicts a MTT assay of H1299 Non small lung cancer cells with PTX, PDG-PTX or PDG as compared to an untreated control. Data is presented as % viable cells.

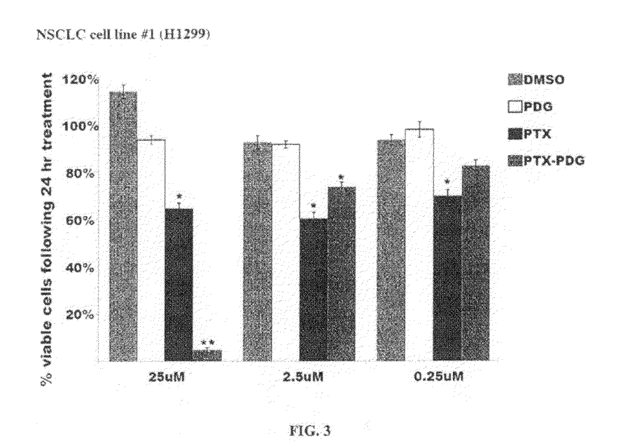

FIG. 4 depicts a MTT assay of H1155 Non small lung cancer cells with PTX, PDG-PTX or PDG as compared to an untreated control. Data is presented as % viable cells.

FIG. 5 depicts a MTT assay of MCF-7 breast cancer cells with PTX, PDG-PTX or PDG as compared to an untreated control. Data is presented as % viable cells.

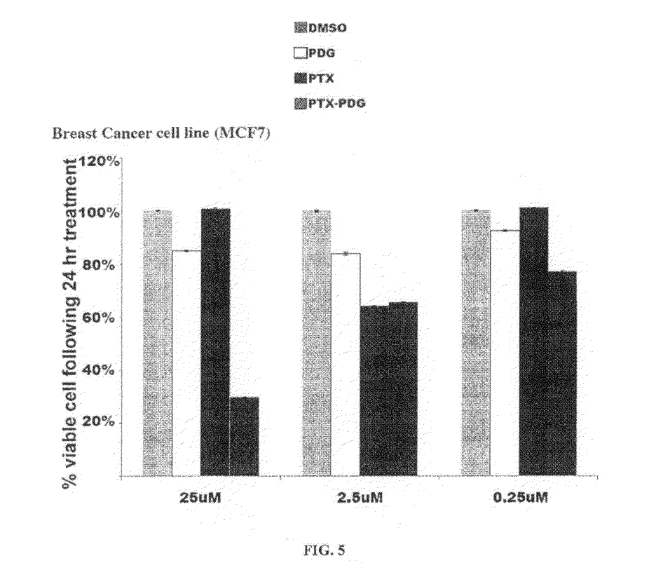

FIG. 6 depicts trends in binding affinity of lipids as potential conjugates for using albumin as a carrier.

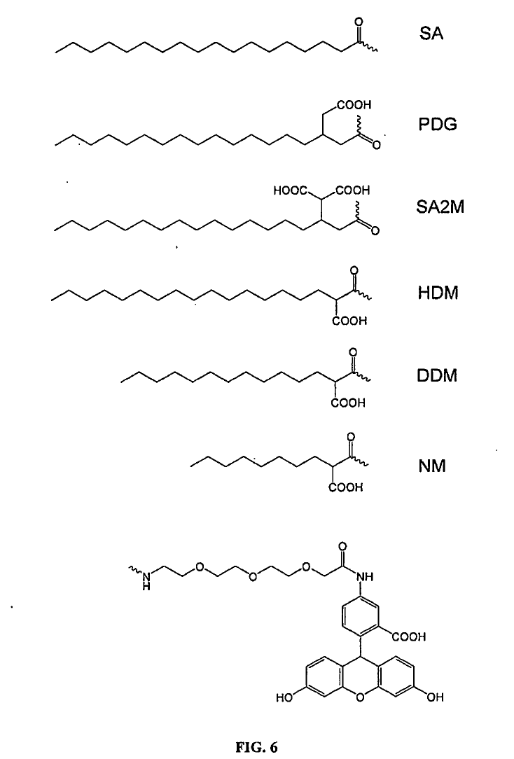

FIG. 7 depicts an Isothermal Titration Calorimetry data for the binding of stearic acid-fluoresceinamine to human serum albumin.

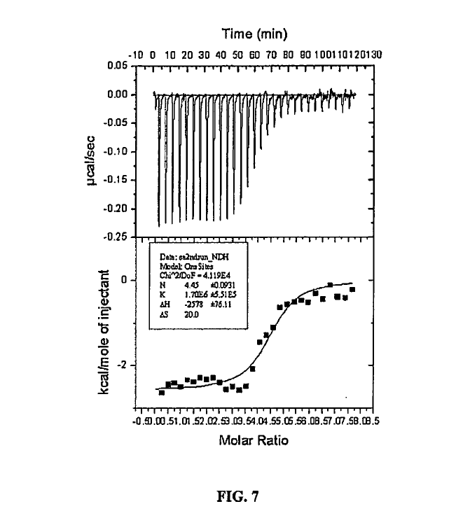

FIG. 8 depicts an Isothermal Titration Calorimetry data for the binding of PDG-fluoresceinamine to human serum albumin.

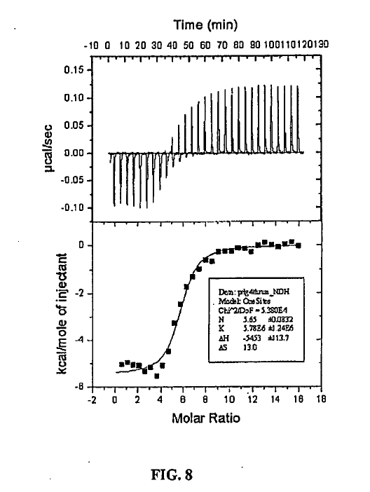

FIG. 9 depicts two structural isomers of a PTX conjugate. Structures show that when counting from the carboxylate, compound 2 branches at the alpha position, and compound 3 at the beta position.

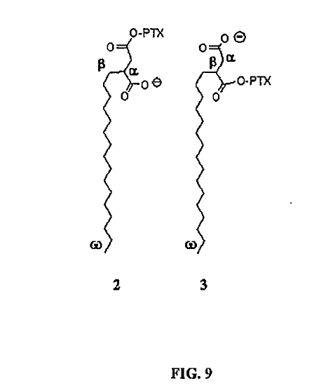

### DETAILED DESCRIPTION

New drug formulations and methods of their use are provided herein. The new formulations described herein are hypothesized to utilize three in vivo properties and functions of serum albumin. First, albumin circulates for a long time with t_{1/2} of 19 days in humans. Secondly, it carries tightly bound fatty acids. Thirdly, when the drug formulation is a cancer therapy and when the tumor is growing, the protein accumulates and degrades mainly in the tumor. Thus, for example, a stearic acid derivative of paclitaxel could possess prolonged circulatory t_{1/2} as bound to albumin and result in significant accumulation of the drug in the solid tumors.

Albumin and IgG are attractive drug carriers not only because drug molecules latched on these proteins can enjoy a long circulatory life but also because there exists a strong possibility that drug bound to these proteins can be delivered to solid tumor or inflammatory tissue. The binding affinity in the case of fatty acids to albumin originates from two sources: one is entropy (ΔS)-driven hydrophobic interaction between the long alkyl chain of fatty acid and the binding cavity and the other is enthalpy (ΔH)-driven electrostatic attraction between the carboxylic acid anion and positive charges from Lys and Arg in the periphery of the binding pocket. Because of the high affinity of fatty acids toward albumin, a variety of drugs have been modified with fatty acids (Lambert, 2000). However, in all cases, the —COOH group is directly attached to drug molecules because chemistries involved are often straightforward. An important drawback in this simple approach is that the conjugate will bind albumin with a lower affinity than free fatty acids because the electrostatic contribution is no longer available in the interaction of the conjugate with fatty acid binding site. The technology described herein involves both enthalpy as well as entropy contributions to albumin bind of the conjugate. There are reports of an unbound carboxylate anion (Ekrami, Kenney, and Shen, 1995; Kurtzhals, Havelund, Jonassen et al. 1995).

The present formulations and processes utilize the conjugation chemistry described herein. Importantly, the —COOH function of the fatty acid remains intact in the final conjugate. Preferably, the fatty acid molecule has more than one carboxylic acid moiety. When the fatty acid molecule contains more than one carboxylic acid moiety, the preferred form of the molecule is the anhydride. Preparation of anhydrides from dicarboxylic acids is well known in the art.

In one embodiment, the present disclosure is directed to a conjugate composition comprising a long-chain fatty acid and a drug, wherein said conjugate has at least one free carboxylic acid or carboxylate group from said fatty acid. Preferably, the fatty acid is a dicarboxylic acid. More preferably, the fatty acid is derived from an anhydride. As used herein, the term “fatty acid” refers to C₁₀₋₂₅ alkyl fatty acids and derivatives, as well as anhydrides of dicarboxylic acids. Preferably, the alkyl chain has from 12 to 20 carbons. More preferably, the alkyl chain has from 14 to 16 carbons. Useful fatty acids include any alkyldioic acid, which is a straight alkyl chain with carboxylates at the distal ends which include; malonic acid, succinic acid, glutaric acid, adipic acid, pimelic acid, suberic acid, azelaic acid, sebacic acid and the like. When a polycarboxylic acid with a long alky chain is used in derivatizing the drug molecule, the resulting conjugate will carry a free —COOH group. Dicarboxylic acids such as malonic, succinic, and glutaric acids are useful and their simple derivatives that contain one long alkyl chain in which the number of carbons varies from 8 to 25, preferably 8 to 20, 10 to 20, 12 to 20 or 14-16. In addition to these dicarboxylic acid derivatives, any compounds that contain three or more —COOH groups can be used for the same purpose. Examples may include; citric acid, tricarboxylic acid and its derivatives such as beta-methyltricarboxylic acid, and 1,2,3,4-butanetetracarboxylic acid. Cyclic dicarboxylic acids such as camphoric acid and cyclic 1,3,5-cyclohexanetricarboxylic acid can also serve the same purpose. Mixed di- or multi-acids containing an inorganic acid are also included; a naturally occurring example is phosphorylated N-acetyltyrosine, while sulfate esters of a hydroxy-containing carboxy acid is an example of synthetic in origin.

Most preferably, the fatty acid is glutaric acid, in particular 3-pentadecylglutaric anhydride (PDG). The composition can further comprise a pharmaceutically acceptable excipients or diluents.

Any drug or compound can be used in the conjugate so long as the drug or compound has a nucleophilic group or can be modified to contain a nucleophilic group. These drugs could be chemical in nature such as an NSAID e.g. naproxen, acetaminophen, or ibuprofen. They could also be used for slow release of analgesics, such as after surgery. Typical analgesics could be codeine, oxycodone and morphine. However, the present conjugation technology is not limited to small molecule drugs. It can be used to modify proteins or peptides as these contain many amino acid nucleophiles that can react with the long-chain fatty acid. Additionally, most non-circularized peptides have carboxyl and amino termini, both of which could be used to react with the long-chain fatty acid. The carboxyl end would require the synthesis of a promoiety such as an ethanolamine linker. The formed ester would be labile making it a prodrug and the primary amine could then be reacted with the long-chain fatty acid under the usual conditions. Typical examples of natural peptides could be hormones e.g. Insulin. Other useful controlled release formulations could include short multi-dose regimens such as antibiotics or steroids. Controlling the release rate of these drugs could be done using multiple different conjugates, some possessing higher affinity and some possessing lower. The lower affinity conjugates would come off quicker allowing a burst while the higher affinity conjugates would come off slower allowing steady state concentration maintenance. Some possible antibiotics include cephalexin, amoxicillin and vancomycin as well as many others known in the art. Some possible steroids include prednisone, hydrocortisone and budesonide as well as most corticosteroids. In a preferred embodiment, the drug is paclitaxel.

In an embodiment, the present disclosure is directed to a method of preparing the long-chain fatty acid conjugate of claim 1 comprising contacting said drug with said long-chain fatty acid wherein said conjugate is prepared. In an aspect of this embodiment, a method of conjugation is via a lipid which allows a facile single-pot synthesis of conjugate from drug. In particular, 3-pentadecylglutaric anhydride (PDG) chemistry allows for any drug containing a nucleophile to potentially be conjugated as described herein. By nucleophile it is meant an alcohol, a thiol, a primary amine or secondary amine. These nucleophiles can be synthesized on the drug. For example, nucleic acids such as antisense and siRNA are synthesized with C6 amino modifiers imparting a free amine to the drug. In this case, the drug would now readily be able to react with PDG. These modifications can be reversible thus generating prodrugs which will readily react with PDG that can be subsequently cleaved back to the intact drug. As an example, carboxylic acids are non-nucleophilic but common to many drugs. The formation of an ester using ethanolamine would yield a prodrug through a readily cleavable ester, as well as imparting a free amine which could react with PDG.

The terms “alkenyl” and “alkylenyl”, when used alone or in combination, embraces linear or branched radicals having at least one carbon-carbon double bond in a moiety having between two and ten carbon atoms. Examples include, without limitation, ethenyl, propenyl, alkyl, propenyl, butenyl and 4-methylbutenyl. The terms “alkenyl” and alkylenyl embrace radicals having “cis” and “trans” orientations, or alternatively, “E” and “Z” orientations, as appreciated by those of ordinary skill in the art.

The following schemes 1-5 depict synthetic routes for preparing fatty acid molecules or conjugates as described herein. Scheme 1 depicts a synthetic route for preparing PDG (3-pentadecylglutaric anhydride).

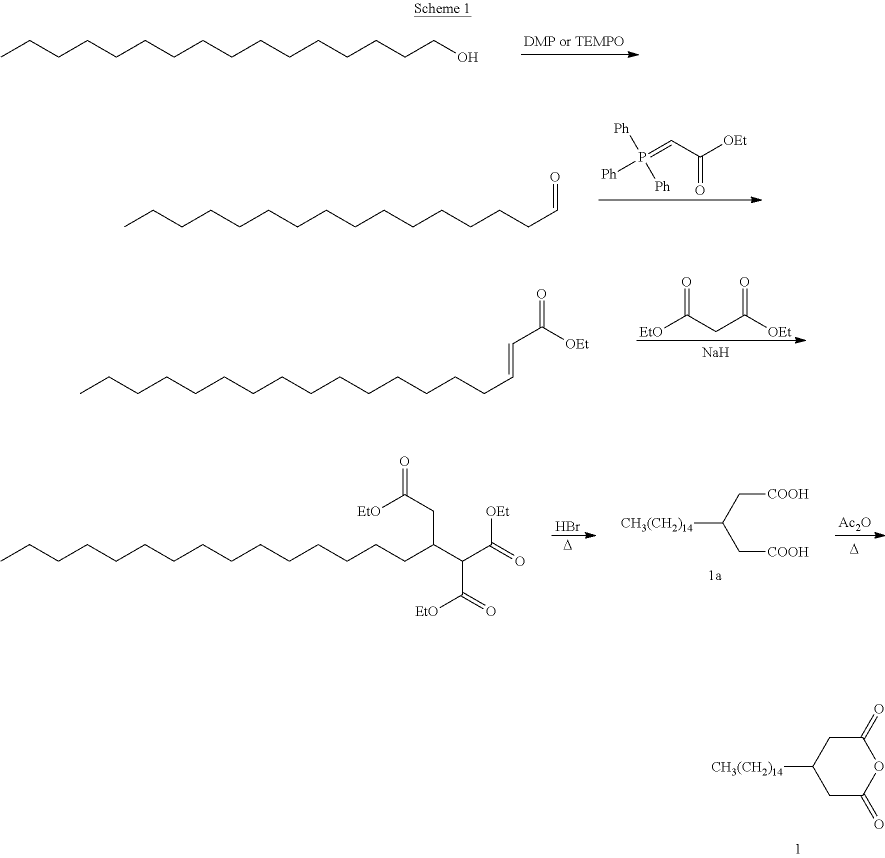

Scheme 2 depicts a synthetic route of PDG-PTX (paclitaxel).

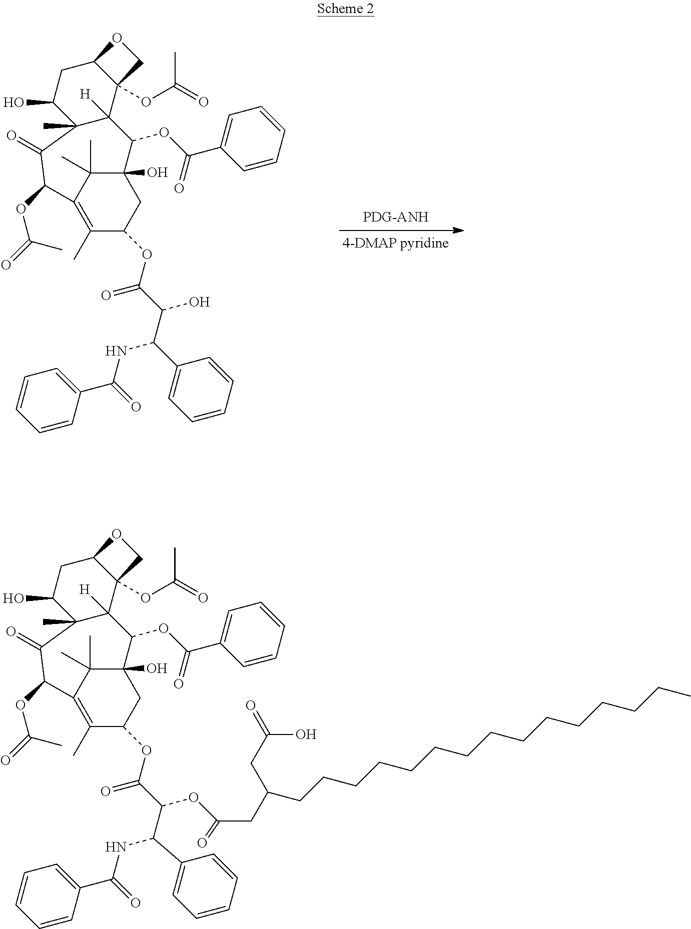

Scheme 3 depicts a one-step synthesis of a paclitaxel-fatty acid conjugate.

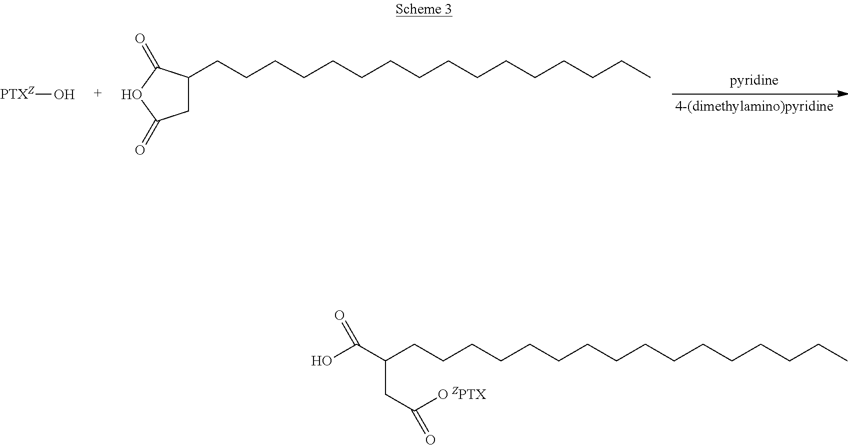

Scheme 4 depicts a synthetic route of PDG-DOX (doxorubicin).

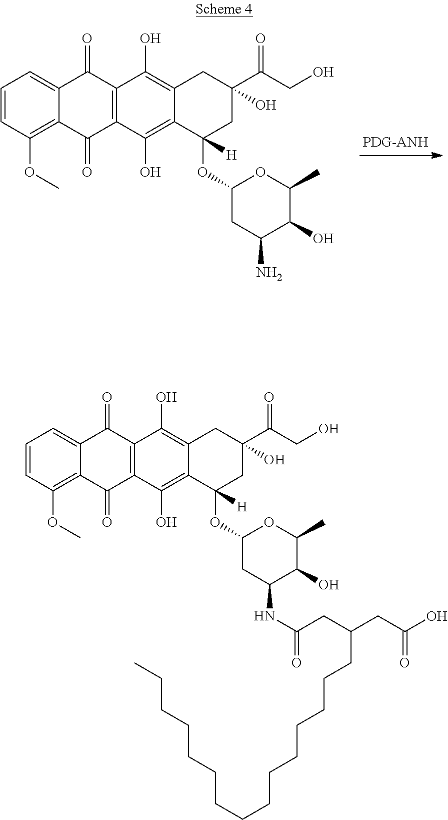 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; CCCCCCCCCCCCCCCC(CC(=O)O)CC(=O)N[C@H]1C[C@H](O[C@H]2C[C@](O)(C(=O)CO)Cc3c(O)c4c(c(O)c32)C(=O)c2c(OC)cccc2C4=O)O[C@@H](C)[C@H]1O]

Scheme 5 depicts a synthetic route of PDG-GEM (gemcitabine).

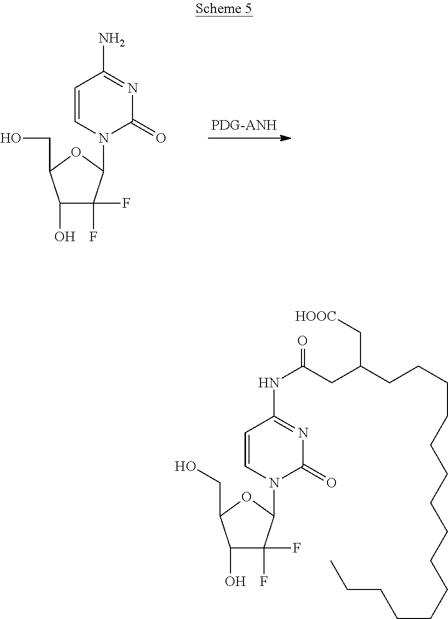 [SMILES: Nc1ccn(C2OC(CO)C(O)C2(F)F)c(=O)n1 ; CCCCCCCCCCCCCCCC(CC(=O)O)CC(=O)Nc1ccn(C2OC(CO)C(O)C2(F)F)c(=O)n1]

The compositions and methods of the invention can be used to prepare cancer therapies, particularly drug formulations directed at solid tumors. The natural tendency of serum albumin to aggregate in growing tumors can be utilized to generate useful antineoplastic formulations by long-chain fatty acid conjugation. Suitable drugs for use in the drug formulations of the present invention include anti-mitotic agents (e.g. Paclitaxel, docetaxel), topoisomerase ii inhibitors (e.g. Doxorubicin, daunorubicin, epirubicin) and nucleic acid analogs (e.g. Gemcitabine, 5-fluorouracil, methotrexate). Any drug designed to target and kill rapidly dividing cells that has a free nucleophile, or can be modified to contain a free nucleophile, could be formulated using a long-chain fatty acid for increased efficacy.

In a non-tumor bearing state, albumin circulates with a half-life of about 19 days. Lipids with high affinity dissociate from albumin slowly while low affinity lipids dissociate at a higher rate. Thus the affinity of the conjugate for albumin can be modulated to generate a drug-specific controlled release formulation.

Different formulations will require conjugates of different affinities which can be modulated through several mechanisms. It is likely that affinity will decrease as the tail length of the fatty acid decreases and the affinity will increase as the tail length increases up to about 22 carbons. Longer chain carbon tails can still be useful. The affinity also appears dependent on the presence of an anionic charge at the head of the molecule putatively due to electrostatic interactions with lysines and arginines on the surface of the binding pocket on albumin. Removal of the charge altogether will decrease affinity while increasing the number of charges or the flexibility of the arm bearing the charge could increase the affinity. The flexibility of the molecule can influence affinity, for example, the flexibility of the 3 carbon symmetrical head group, malonic acid, can be different than the more flexible 5 carbon symmetrical head group, glutaric acid. However, each of these allows maintenance of one anionic charge. The more flexibility to orient the charge at the surface of the pocket can increase affinity. Similarly, a 4 carbon asymmetrical head group, such as succinic acid, is useful. The succinic acid can also be cyclized into an anhydride allowing facile conjugation with nucleophiles. A succinic acid based lipid as well as any other alkyldioic acid could be used as a head group for the formulation of lipid drug conjugates for the purpose of binding albumin. By alkyldioic acid is meant a straight alkyl chain with carboxylates at the distal ends which include; malonic acid, succinic acid, glutaric acid, adipic acid, pimelic acid, suberic acid, azelaic acid, sebacic acid and the like.

In another embodiment, the present disclosure is directed to a method of solubilizing a drug comprising contacting the long-chain fatty acid-drug conjugate described herein with a medium in which said conjugate is to be solubilized. The medium can be any liquid. Preferably, the medium is serum. In a preferred embodiment, the conjugate has a 100-fold increase in solubility compared to the solubility of the drug alone in a particular medium. More preferably, the conjugate has a 200-fold increase in solubility. Most preferably, the conjugate has a 250-fold increase in solubility.

In another embodiment, the present disclosure is directed to a compound having the structure

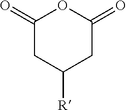

wherein R′ is a substituted or unsubstituted C₁₀₋₂₅ alkyl or C₁₀₋₂₅ alkylenyl. Preferably, R′ is C₁₂₋₂₀ alkyl. More preferably, R′ is C₁₄₋₁₆ alkyl. Most preferably, the compound is 3-pentadecylglutaric anhydride.

The drug formulations for cancer can be used particularly in the treatment of solid tumors. “Tumor,” as used herein, refers to all neoplastic cell growth and proliferation, whether malignant or benign, and all pre-cancerous and cancerous cells and tissues. The term “solid tumor” refers to a cancer or carcinoma of body tissues other than blood, bone marrow, and lymphoid system. Examples of cancers that are classified as solid tumors include but are not limited to lung cancer, breast cancer, ovarian cancer, colon cancer, liver cancer, gastric cancer, prostate, and skin cancer. The present invention is directed to methods for treating human subjects with solid tumors that comprise CD40-expressing carcinoma cells, including, but not limited to, ovarian, lung (for example, non-small cell lung cancer of the squamous cell carcinoma, adenocarcinoma, and large cell carcinoma types, and small cell lung cancer), breast, colon, kidney (including, for example, renal cell carcinomas), bladder, liver (including, for example, hepatocellular carcinomas), gastric, cervical, prostate, nasopharyngeal, thyroid (for example, thyroid papillary carcinoma), and skin cancers such as melanoma, and sarcomas (including, for example, osteosarcomas and Ewing's sarcomas).

The invention will now be further described by way of the following non-limiting examples.

### EXAMPLES

### Example 1

### Synthesis of PDG-paclitaxel

A 50 mg (0.15 mmol, 2 Eq) amount of 3-pentadecylglutaric anhydride (PDG), 64 mg (0.075 mmol, 1 Eq) paclitaxel and 0.9 mg (0.0075 mmol, 0.1 Eq) of 4-dimethylaminopyridine was added to a 1 dram vial equipped with a small stir bar. To the dry powders was added 1.3 mL of freshly distilled pyridine at room temperature with stirring. The reaction mixture was capped with a silicone/PTFE septum and the atmosphere was replaced with argon. The reaction mixture was allowed to react overnight (18 h) at room temperature at which point TLC using EtOAc as the eluent showed a complete disappearance of paclitaxel (R_{f}=0.9) and the emergence of a new UV active spot exhibiting minor tailing (R_{f}=0.3-0.5). The reaction mixture was taken up in ˜20 mL dichloromethane and subsequently washed three times with ˜20 mL acidified brine to remove the pyridine and DMAP. The DCM layer was dried over MgSO₄, filtered and concentrated then taken up in EtOAc. The mixture was adsorbed onto a silica column and eluted with isocratic EtOAc. When concentrated, 75 mg of the product was recovered as white crystals. (85% yield).

### Example 2

### Measurement of the Solubility of the Stearic Acid Derivative of Paclitaxel

A 37.5 mg amount of PDG-PTX was dissolved in 5 mL dry EtOH to yield a 7.5 mg/mL solution. This solution was then spiked with (3.14 μL/3.5×10⁶ DPM/1.43×10⁻⁴ μmol/168 ng) [³H-PDG-PTX] from Moravek. Various amounts of the solution were added to 5 vials in increasing amounts; 50 μL, 500 μL, 1000 μL, 1500 μL, 1950 μL. The EtOH was then evaporated from all vials via nitrogen and replaced with 5 mL of PBS containing increasing concentrations of HSA; 0%, 1%, 2%, 3%, 4%. The higher concentrations of albumin were added to the vials containing more PDG-PTX. The suspensions were shook at 25° C. and 1 mL aliquots were removed at various timepoints; 24 h, 48 h, 72 h, 192 h (8 d), 336 h (14 d). The aliquots were filtered through 0.1 μm inorganic membranes and analyzed via liquid scintillation counting to quantify the PDG-PTX, as well as BCA assay to quantify the concentration of HSA.

It was found the dissolution of the crystals was slow. Thus, the crystals were dissolved in t-BuOH and added to a stirred solution of 1% human serum albumin at a rate of 0.25 μL/min. The suspension was then lyophilized overnight to remove the water and t-BuOH, and the cake was reconstituted in dH₂O to regenerate a 1% human serum albumin solution. The solution was filtered and analyzed via scintillation counting showing a solubility of about 825 μM of PDG-PTX. This approximates to about 5.5 molecules of PDG-PTX to each molecule of albumin in a 1% solution. Albumin has a theoretical binding limit of 6 molecules so the albumin is nearly saturated with conjugate.

### Example 3

### Comparison of the in vitro cytotoxicity of PDG-PTX versus PTX alone in NSCLC, Breast and Ovarian Cancer Cell Lines

The cytotoxicity of PDG, PTX and PDG-PTX was assayed across 4 cell lines; H1299 (NSCLC), H1155 (NSCLC), Es2 (Ovarian), MCF7 (breast). The literature suggested an IC₅₀ for PTX around 25 μM (MDA-MB-231, SK-Br3) so concentrations of 0.25, 2.5 and 25 μM were used. Stock solutions of 2.5 mM (100×) PDG, PTX and PDG-PTX were made in DMSO. Then at about 60-70% confluency, to the cell media was added 0.1, 1 or 10 μL of the stock in DMSO. To keep everything constant, the 0.1 and 1 μL aliquots were made up to a volume of 10 μL in DMSO. The control cells were treated only with 10 μL of DMSO. The cells were incubated for 24 h with the drug, washed, then incubated with MTT for 30 minutes and the formazan crystals dissolved in DMSO and quantified (λ=560) via UV.

### Example 4

### Measurement of the Increased Half Life of Paclitaxel in the Bloodstream by Measuring the Pharmacokinetics in a Mouse Model

Formulation of PDG-PTX: An equivalent dose of 3 mg PTX and 225 μL [³H-PDG-PTX] (0.5 mCi/mL, 11 Ci/mmol) will be concentrated then dissolved in 250 μL of t-BuOH and added to 2.5 mL of 4% delipidated HSA in PBS at a rate of 10 μL/min. The resulting solution will be lyophilized overnight and reconstituted with 2.5 mL dH₂O. The solution will be injected into the mice via the tail vein at a dose of 0.12 mg/mouse (˜6 mg PTX/kg mouse). (0.12 mg/1×10⁷ DPM/0.1 mL PBS/20 g mouse).

Formulation of Taxol-Like PTX: An equivalent dose of 3 mg PTX and 114 μL [³H-PTX] (0.25 mCi/0.25 mL, 36 Ci/mmol) will be concentrated then dissolved in 0.1 mL (1:1 v/v) Cremophor EL and absolute EtOH at a concentration of 30 mg/mL. This solution will be diluted 5 fold to 6 mg/mL with PBS just prior to injection. The dose will be 20 μL (˜6 mg PTX/kg mouse) administered via the tail vein. (0.12 mg/1×10⁷ DPM/0.02 mL PBS/20 g mouse)

Pharmacokinetic experiment: Female Balb/c mice (6-8 weeks old) will be shaved on the right flank and 2×10⁵ CT-26 tumor cells in 100 μL of media will be injected subcutaneously using a 1 mL syringe and 25 G needle. The pharmacokinetic study will begin when all mice have tumors between 50-100 mm³ (L²*W) where L is the longer measurement (˜9 days post inoculation). The mice will be injected with 1 of 3 treatments: taxol-like, C₁₈-PTX, PDG-PTX. The mice will be sacrificed in triplicate at a given timepoint. The taxol-like formulation will be cleared quickly thus the timepoints will only extend out to 24 h; 0.25, 1, 2, 4, 8, 12, 24 hours. The C₁₈-PTX and PDG-PTX formulation are expected to enjoy longer half-lives and will have data points collected out to 72 h; 0.25, 1, 3, 6, 18, 36, 72 hours. A few minutes prior to sacrifice the mice will be injected i.p. with 10 mg ketamine (1 mL syringe 25 G needle). Once unconscious, the mice will be cut open and blood collected via cardiac puncture (1 mL syringe 25 G needle) with subsequent termination. The blood will be aliquoted as 200 μL in triplicate and the rest stored at −20° C. The organs will be harvested from the deceased mice, blotted dry and small (˜100 mg) pieces excised, weighed and stored in scintillation vials in triplicate. The rest of the organ will be stored at −20° C. until further use if warranted. Specifically the organs will include the liver, lungs, heart, kidneys, spleen, tumor and injection site.

Blood Processing: An aliquot of 200 μL whole blood will be added to scintillation vials in triplicate with 1 mL of Solvable®. The samples will be incubated for 1 hour at 55° C. turning it brownish-green. To this will be added 100 μL of 0.1 M EDTA di-sodium followed by 300 μL of 30% H₂O₂ in 100 μL aliquots. The reaction can be quite vigorous and will be allowed to complete over 30 minutes RT. The vial will then be capped and heated at 55° C. for an additional hour which should yield a clear to slightly yellow solution. The sample will be cooled to RT and 15 mL of Ultima Gold will be added and mixed thoroughly. The samples will sit in the LSC for 1 hour to adapt to light and temperature prior to counting.

Organ Processing: A small chunk of the organs will be added to scintillation vials in triplicate (˜100 mg) and 1 mL of Solvable® will be added. The samples will be heated to 55° C. for 2 hours with occasional swirling/venting. Once dissolved (slightly yellow) the samples will be cooled to RT and 200 μL of 30% H₂O₂ will be added in 100 μL aliquots. The samples will then be heated to 55° C. again for 30 minutes. The samples will be cooled to RT and 10 mL Ultima Gold added to each sample with thorough mixing. The samples will sit in the LSC for 1 hour to adapt to light and temperature prior to counting.

Quench Curve Correction: A normal mouse will be anesthetized with ketamine and its blood removed via cardiac puncture. The mouse will be sacrificed and the major organs harvested. Aliquots of 175 μL, 200 μL and 225 μL of blood will be spiked with a known specific activity of PDG-PTX (−1×10⁴ DPM). Additionally, ˜75, 100 and 125 mg samples of organs will be spiked with a known specific activity of PDG-PTX (˜1×10⁴ DPM). The samples will then be processed the same way as the aforementioned analyses and compared to a triplicate of PDG-PTX in scintillation cocktail. (˜1×10⁴ DPM) The counting efficiency will be the ratio of organ DPM/standard DPM.

### Example 5

### Increased Delivery of Paclitaxel to Tumors by Measuring Pharmacodynamics

Treatment groups of 4 female BALB/C mice 6-8 weeks of age will be randomly separated and inoculated with 2×10⁵ CT-26 cells in 100 μL of media on the right hind legs. Once the tumors grow to about 50-100 mm³ (L²*W) the mice will be treated with 4 mg/kg equivalents of paclitaxel formulated as “taxol-like” and formulated as PDG-PTX complexed with serum albumin. We will also test increasing doses of PDG-PTX to determine the maximally tolerated dose (MTD) which we expect to be higher than paclitaxel formulated as taxol-like. The mice will be treated with 3 doses of the formulation and the size of the tumor will be measured daily using vernier calipers. The experiment will end when the tumors are too large for IACUC standards and the mice will be sacrificed.

### Example 6

### Increased Binding Affinity to Albumin of Conjugates Bearing a Negative Charge

The lipid affinities for albumin are tested by synthesizing a short polyethyleneglycol linker between the lipid of concern and a fluorophore, fluoresceinamine, which acts as a drug surrogate. The lipid-fluoresceinamine conjugate is then titrated into a solution of serum albumin in PBS and the heat of interaction is measured using an isothermal titration calorimeter. From this the enthalpy of association can be calculated yielding and subsequently a binding affinity can be calculated.

### Example 7

### Synthesis of α-carboxymethylstcaric acid conjugate of PTX

The synthesis of the α-carboxymethylstearic acid conjugate of PTX is based on a previously reported and commonly used synthesis using succinic anhydride. (Thierry B, Kujawa P, Tkaczyk C, et al. 2005: PMID 15700982) A 100 mg amount of paclitaxel (0.117 mmol) was weighed and added it to a dry reaction vessel. To this was added 76 mg of hexadecyl succinic anhydride (0.234 mmol). The solids were dissolved in 1.2 mL dry pyridine containing 1.4 mg 4-(dimethylamino)pyridine (0.012 mmol). The reaction vessel was closed, purged with nitrogen and stirred for 48 h at room temperature. TLC in 100% ethyl acetate confirmed the disappearance of paclitaxel had plateaued with the appearance of a new product. The product was purified via liquid chromatography by eluting with 7:3:1 petroleum ether: ethyl acetate: acetic acid. Using this solvent system, the R_{f} of the product is 0.5 on TLC. The pure product was obtained as a white solid (72 mg, 52% yield); ¹H NMR 300 MHz (CDCl₃) δ8.12; (2H, d), 7.75; (2H, t), 7.60; (1H, q), 7.54-7.26; (8H, m), 6.27; (1H, s), 6.17; (1H, t), 5.95; (1H, m), 5.67; (1H, d), 5.50; (1H, m), 4.94; (1H, d), 4.41; (1H, m), 4.83; and 4.16; (2H, dd), 3.77; (1H, d), 2.90-1.98; (8H, m), 2.20; (4H, d), 1.98-1.72; (4H, m), 1.72-1.62; (2H, s), 1.62-1.38; (2H, dm) 1.34-0.96; (25H, br m), 0.88; (3H, t). Mass spectrometry LC/MSD Trap SL; (M+H)⁺=1178.8 with Intensity 0.9×10⁹, (M+Na)⁺=1201.1 with Intensity=4.7×10⁹.

### Example 8

### Solubility/Albumin affinity of α-carboxymethvlstearic acid conjugate of PTX

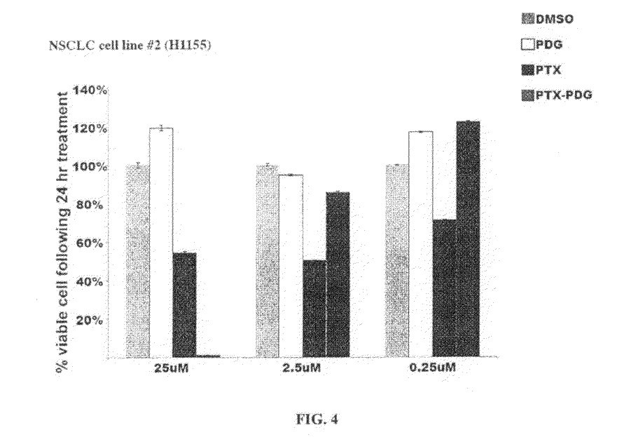

To generate the solubility profile, a 40% solution of delipidated Bovine Serum Albumin will be made and saturated with the a-carboxymethylstearic acid conjugate of PTX at 25° and 37°. The precipitate will be filtered and the absorbance read at 227 nm subtracting the absorbance of PBS and albumin. The 40% solution will be serially diluted to 20%, 10%, 5% and 2.5% with filtration at each step. The absorbance will be read for each solution at each temperature and plotted. A standard curve of the conjugate showed the conjugation did not affect absorbance. This reaction will generate two structural isomers. One product will be a nineteen carbon fatty acid where paclitaxel is esterified at the β carbon with respect to the carboxylic acid. The other product will be an eighteen carbon fatty acid where branching occurs at the α position of the fatty acid. (FIG. 4) It is postulated that this a product will bind albumin with slightly better affinity due to the extra carbon off the main chain allowing paclitaxel to remain out of the pocket. Sterically, we also postulate the a product to be the major product due to some steric hindrance at the carboxyl carbon adjacent to the acyl chain. Both products will be isolated using liquid chromatography and each component will be assayed individually.

There are several methods to measure albumin affinity. A 40% solution of albumin in PBS will be prepared while adding varying amounts of the conjugate in PBS to two wells of a microdialysis chamber. After equilibration at 37° C., the results of the non albumin chamber can be read via UV absorption. Then the bound and unbound fractions can be calculated and the scatchard analysis used to analyze the data. (Cho, Mitchell et al. 1971) Alternatively, if solubility permits the binding affinity can be measured directly using a microcal VP-ITC calorimeter. This technique has already demonstrated its effectiveness in measuring the affinity of fatty acids to albumin. (Fang, Tong, Means 2006 PMID: 16413837)

### Example 9

### Pharmacokinetic Studies of α-carboxymethylstearic acid conjugate of PTX

Pharmacokinetic studies on the conjugate will require the use of a tag for quantification such as ¹⁴C-paclitaxel from Sigma-Aldrich (product number: P1598). Initially the mice will be innoculated on the flank and the tumors allowed to grow to ˜100 mm³. At this point, a single dose of 4.2 mg/kg with respect to paclitaxel will be administered as a complex with albumin, as compared to Abraxane® phase I clinical trials. (Desai 2001) Preferably, the samples would have at least 10⁶ dpm so they are roughly 1000 fold higher than background. Blood samples will be taken 0.5, 2, 12, 24, 48 and 72 hours post injection. Paclitaxel concentrations will be quantified through direct liquid scintillation counting. (Palma and Cho 2007) Pharmacokinetic analysis will be done using a non-compartmental model as was done with Abraxane®. (Desai 2001) To examine the biodistribution, the liver, lungs, heart, spleen and tumor will be homogenized and analyzed for their PTX content.

To monitor the effect of the treatment, we will again innoculate the mice on the flank with B16F10 cells and allow the tumors to progress to ˜100 mm³. At this point, a single dose of 4.2 mg/kg with respect to paclitaxel will be administered as a complex with albumin. The tumor size will be monitored every 2-3 days. Control mice will receive only albumin in PBS. (Palma and Cho 2007)

To test whether FcRn recycling will affect the binding of the conjugate to albumin, soluble FcRn can be generated as has been presented in the literature. (Gastinel Simister Bjorkman 1992 PMID: 1530991; Chaudhury, Brooks et al PMID: 16605266) This binds albumin at an endosomal pH of about 5. Thus to determine if the conjugate will stay bound, the new K_{A} of the conjugate can be measured in the presence of shFcRn at pH 5. As a control, the binding should be done at pH 5 without shFcRn as well.

All documents cited or referenced in the application cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

### BIBLIOGRAPHY

Anderson, C. L., C. Chaudhury, et al. (2006). “Perspective—FcRn transports albumin: relevance to immunology and medicine.” Trends Immunol 27(7): 343-8.
Cho, M. J., A. G. Mitchell, et al. (1971). “Interaction of bishydroxycoumarin with human serum albumin.” J Pharm Sci 60(2): 196-200.
Couvreur P, B. Stella, et al. (2006). “Squalenoyl nanomedicines as potential therapeutics” Nano Lett. 6(11); 2544-2548.
Crown, J., M. O'Leary, et al. (2004). “Docetaxel and paclitaxel in the treatment of breast cancer: a review of clinical experience.” Oncologist 9 Suppl 2: 24-32.
Curry, S., P. Brick, et al. (1999). “Fatty acid binding to human serum albumin: new insights from crystallographic studies.” Biochim Biophys Acta 1441(2-3): 131-40.
Ekrami, H. M., A. R. Kennedy, and W. C. Shen (1995). “Water-Soluble Fatty Acid Derivatives as Acylating Agents for Reversible Lipidizatio of Polypeptides.”FEBS Lett 371:283-286.
Gralow, J., H. Rugo, et al. (2008). “Novel taxane formulations in the treatment of breast cancer: a thought leader discussion and consensus roundtable.” Clin Breast Cancer 8(1): 33-7.
Huan M. L, S. Y. Zhou et al. (2009) “Conjugation with Alpha-linolenic Acid Improves Cancer Cell Uptake and Cytotoxicity of Doxorubicin.” Bioorg Med Chem Lett. 19(9);2579-84.
Jain, R. K. (2005). “Normalization of tumor vasculature: an emerging concept in antiangiogenic therapy.” Science 307(5706): 58-62.
Kim, J., C. L. Bronson, et al. (2006). “Albumin turnover: FcRn-mediated recycling saves as much albumin from degradation as the liver produces.” Am J Physiol Gastrointest Liver Physiol 290(2): G352-60.
Kratz, F., R. Muller-Driver, et al. (2000). “A novel macromolecular prodrug concept exploiting endogenous serum albumin as a drug carrier for cancer chemotherapy.” J Med Chem 43(7): 1253-6.
Kurtzhals, P. (2007). “Pharmacology of insulin detemir.” Endocrinol Metab Clin North Am 36 Suppl 1: 14-20.
Lambert, D. M. (2000). “Rationale and applications of lipids as prodrug carriers.” Eur J Pharm Sci 11 Suppl 2: S15-27.
Maeda, H. (2001). “The enhanced permeability and retention (EPR) effect in tumor vasculature: the key role of tumor-selective macromolecular drug targeting.” Adv Enzyme Regul 41: 189-207.
Manoharan, M., G. B. Inamati et al. (2002). “Improving Antisense Oligonucleotide Binding to Human Serum Albumin: Dramatic Effect of Ibuprofen Conjugation.” ChemBioChem 3(12):1257-1260.
Paal, K., J. Muller, et al. (2001). “High affinity binding of paclitaxel to human serum albumin.” Eur J Biochem 268(7): 2187-91.
Palma, E. and M. Cho (2007). “Improved systemic pharmacokinetics, biodistribution, and antitumor activity of CpG oligodeoxynucleotides complexed to endogenous antibodies in vivo.” Journal of Controlled Release 120: 95-103.
Palma, E., D. G. Klapper, et al. (2005). “Antibodies as drug carriers III: design of oligonucleotides with enhanced binding affinity for immunoglobulin G.” Pharm Res 22(1): 122-7.
Poldy J, R. Peakall et al. (2008). “Pheromones and analogs from Neozeleboria wasps and the orchids that seduce them: a versatile synthesis of 2,5-dialkylated 1,3-cyclohexanediones” Tetrahedron Lett. 49(15): 2446-2449.
Rehlaender, B. N. and M. J. Cho (2001). “Anti-drug antibodies as drug carriers. I. For small molecules.” Pharm Res 18(6): 745-52.
Rehlaender, B. N. and M. J. Cho (2001). “Antibodies as drug carriers. II. For proteins.” Pharm Res 18(6): 753-60.
Sandanaraj, B. S., H. Gremlich, et al. (2009). “Fluorescent Nanoprobes as a Biomarker for Increased Vascular Permeability: Implications in Diagnosis and Treatment of Cancer and Inflammation.” Bioconjugate Chem (in print).
Singh S. and A. K. Dash (2009). “Paclitaxel in Cancer Treatment: Perspectives and Prospects of Its Delivery Challenges.” Crt Rev Ther Drug Carrier Syst 26(4):333-372.
Spector, A. A. (1975). “Fatty acid binding to plasma albumin.” J Lipid Res 16(3): 165-79.
Stehle, G., A. Wunder, et al. (1998). “Albumin synthesis rates in cachectic cancer patients with an ongoing acute-phase protein response.” Ann Surg 228(5): 720.
Stehle, G., A. Wunder, et al. (1999). “Albumin-based drug carriers: comparison between serum albumins of different species on pharmacokinetics and tumor uptake of the conjugate.” Anticancer Drugs 10(8): 785-90.
Tanaka, T., S. Shiramoto, et al. (2004). “Tumor targeting based on the effect of enhanced permeability and retention (EPR) and the mechanism of receptor-mediated endocytosis (RME).” Int J Pharm 277(1-2): 39-61.
Vaishampayan, U., R. E. Parchment, et al. (1999). “Taxanes: an overview of the pharmacokinetics and pharmacodynamics.” Urology 54(6A Suppl): 22-9.
Wolff, A. C., R. C. Donehower, et al. (2003). “Phase I study of docosahexaenoic acid-paclitaxel: a taxane-fatty acid conjugate with a unique pharmacology and toxicity profile.” Clin Cancer Res 9(10 Pt 1): 3589-97.
Wolfrum, C., S. Shi et al. (2007). “Mechanisms and Optimization of In Vivo Delivery of Lipophilic siRNAs.” Nature Technol 25(10):1149-1157.

What is claimed is:

1. A conjugate composition comprising a long-chain fatty acid and a drug, wherein said conjugate has at least one free carboxylic acid or carboxylate group from said fatty acid.

2. The composition of claim 1, wherein said fatty acid is a dicarboxylic acid.

3. The composition of claim 1, wherein said fatty acid is derived from an anhydride.

4. The composition of claim 1, wherein said drug is a small molecular weight organic compound, (poly)peptide or oligonucleotides.

5. The composition of claim 1, wherein said fatty acid is selected from the group consisting of malonic acid, succinic acid, glutaric acid, adipic acid, pimelic acid, suberic acid, azelaic acid, sebacic acid and their simple derivatives that contain one long alkyl chain in which the number of carbon varies from 8 to 20, citric acid, tricarboxylic acid and its derivatives, beta-methyltricarboxylic acid, and 1,2,3,4-butanetetracarboxylic acid, cyclic dicarboxylic acids, camphoric acid and cyclic 1,3,5-cyclohexanetricarboxylic acid, mixed di- or multi-acids containing an inorganic acid, and phosphorylated N-acetyltyrosine.

6. The composition of claim 1, wherein said fatty acid is glutaric acid.

7. The composition of claim 1, wherein said fatty acid is 3-pentadecylglutaric anhydride.

8. The composition of claim 1, wherein said alkyl chain is a C₁₀₋₂₅ alkyl chain.

9. The composition of claim 1, wherein said alkyl chain is a C₁₂₋₂₀ alkyl chain.

10. The composition of claim 1, wherein said alkyl chain is a C₁₄₋₁₆ alkyl chain.

11. The composition of claim 1, wherein said drug contains a nucleophilic group or can be modified to contain a nucleophilic group.

12. The composition of claim 1, wherein said drug is PTX (paclitaxel).

13. A method of preparing the long-chain fatty acid conjugate of claim 1 comprising, contacting said drug with said long-chain fatty acid wherein said conjugate is prepared.

14. A method of solubilizing a drug comprising, contacting said long-chain fatty acid-drug conjugate of claim 1 with a medium in which said conjugate is to be solubilized.

15. The method of claim 13, wherein said medium is serum or albumin-containing aqueous solutions.

16. The method of claim 13, wherein said conjugate has a 100-fold increase in solubility compared to said drug alone.

17. A compound having the structure 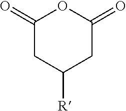
wherein R′ is a substituted or unsubstituted C₁₀₋₂₅ alkyl or C₁₀₋₂₅ alkylenyl.

18. The compound of claim 17, wherein R′ is Cₗ₂₋₂₀ alkyl.

19. The compound of claim 17, wherein R′ is C₁₄₋₁₆ alkyl.

20. The compound of claim 17, which is 3-pentadecylglutaric anhydride.

21. The composition of claim 1, further comprising pharmaceutically acceptable
excipients or diluents.
